SEQUENCE LISTING (58 sequences)

SEQ ID NO: 1 - DNA, 24 bp, Artificial Sequence
  PRIMER
gtgttgtattaggcagagttctcc

SEQ ID NO: 2 - DNA, 24 bp, Artificial Sequence
  PRIMER
ggcagtagatggtgactttatggc

SEQ ID NO: 3 - DNA, 25 bp, Artificial Sequence
  PRIMER
tctcagtcctgatttcttgattttg

SEQ ID NO: 4 - DNA, 21 bp, Artificial Sequence
  PRIMER
ccagagctaacaccacattca

SEQ ID NO: 5 - DNA, 26 bp, Artificial Sequence
  PRIMER
caaccatcagtgatttgatggtttac

SEQ ID NO: 6 - DNA, 23 bp, Artificial Sequence
  PRIMER
gagttgtcactagaaccctgttc

SEQ ID NO: 7 - DNA, 24 bp, Artificial Sequence
  PRIMER
tcctgtaatctgtccccaaacgtc

SEQ ID NO: 8 - DNA, 25 bp, Artificial Sequence
  PRIMER
ttcctcacacaacctattgatagtc

SEQ ID NO: 9 - DNA, 24 bp, Artificial Sequence
  PRIMER
ctgtggaggaaagaaaacactgcc

SEQ ID NO: 10 - DNA, 24 bp, Artificial Sequence
  PRIMER
gcactaaagtagcttgtaactctg

SEQ ID NO: 11 - DNA, 25 bp, Artificial Sequence
  PRIMER
aggaaactgatgtctaaaactatcc

SEQ ID NO: 12 - DNA, 24 bp, Artificial Sequence
  PRIMER
gtgaatccaggaggcagagcttgc

SEQ ID NO: 13 - DNA, 24 bp, Artificial Sequence
  PRIMER
cacagatgcatgggataggggtcc

SEQ ID NO: 14 - DNA, 24 bp, Artificial Sequence
  PRIMER
atggagatgaggtacagtgtgggc

SEQ ID NO: 15 - DNA, 23 bp, Artificial Sequence
  PRIMER
atctctgcagaattcactgggag

SEQ ID NO: 16 - DNA, 23 bp, Artificial Sequence
  PRIMER
gcataggcacacatcacccagag

SEQ ID NO: 17 - DNA, 23 bp, Artificial Sequence
  PRIMER
tcgtaggtgcagacagtaatagc

SEQ ID NO: 18 - DNA, 24 bp, Artificial Sequence
  PRIMER
atgcccacatttctcctccatctc

SEQ ID NO: 19 - DNA, 23 bp, Artificial Sequence
  PRIMER
cgcaggcagaaactccgctgagc

SEQ ID NO: 20 - DNA, 24 bp, Artificial Sequence
  PRIMER
cgtctgagacttccaactgtctgc

SEQ ID NO: 21 - DNA, 23 bp, Artificial Sequence
  PRIMER
aacgttggctcttaccatgcagg

SEQ ID NO: 22 - DNA, 22 bp, Artificial Sequence
  PRIMER
tggactcatggctccctgtcac

SEQ ID NO: 23 - DNA, 27 bp, Artificial Sequence
  PRIMER
atgtatgtaatgtgtacatattggcac

SEQ ID NO: 24 - DNA, 22 bp, Artificial Sequence
  PRIMER
ttggatgcaccccgcttccaca

SEQ ID NO: 25 - DNA, 22 bp, Artificial Sequence
  PRIMER
tctgccatggactctccgtcac

SEQ ID NO: 26 - DNA, 22 bp, Artificial Sequence
  PRIMER
gtttgggattttgggctagctc

SEQ ID NO: 27 - DNA, 22 bp, Artificial Sequence
  PRIMER
atgatgcagtgggagtccgagc

SEQ ID NO: 28 - DNA, 23 bp, Artificial Sequence
  PRIMER
acgccgagctgctcaggactcgc

SEQ ID NO: 29 - DNA, 23 bp, Artificial Sequence
  PRIMER
agcttccctcagggatgagtttg

SEQ ID NO: 30 - DNA, 22 bp, Artificial Sequence
  PRIMER
actcagctcaaatccagggtgc

SEQ ID NO: 31 - DNA, 24 bp, Artificial Sequence
  PRIMER
agcattccgccatctccctagcag

SEQ ID NO: 32 - DNA, 23 bp, Artificial Sequence
  PRIMER
cacccgcaaatcccactagctaa

SEQ ID NO: 33 - DNA, 22 bp, Artificial Sequence
  PRIMER
tccagacttgggactgtggaac

SEQ ID NO: 34 - DNA, 23 bp, Artificial Sequence
  PRIMER
tggaacgttctgtgaccttgcac

SEQ ID NO: 35 - DNA, 23 bp, Artificial Sequence
  PRIMER
ccaggctggtatctgagttgtga

SEQ ID NO: 36 - DNA, 23 bp, Artificial Sequence
  PRIMER
gtgtcacaacggaggacttgctc

SEQ ID NO: 37 - DNA, 24 bp, Artificial Sequence
  PRIMER
tgtgatgcagcggcacaggaaatc

SEQ ID NO: 38 - DNA, 23 bp, Artificial Sequence
  PRIMER
tggtgccttccagtttggacctc

SEQ ID NO: 39 - DNA, 24 bp, Artificial Sequence
  PRIMER
tagggvtcctgctctgcctgatga

SEQ ID NO: 40 - DNA, 23 bp, Artificial Sequence
  PRIMER
agtgctccctcggggttgcattc

SEQ ID NO: 41 - DNA, 23 bp, Artificial Sequence
  PRIMER
gcccagcctcttatgacctattc

SEQ ID NO: 42 - DNA, 23 bp, Artificial Sequence
  PRIMER
tgacagactcctcagcacgagac

SEQ ID NO: 43 - DNA, 23 bp, Artificial Sequence
  PRIMER
ctgctgccaagttgtttctggac

SEQ ID NO: 44 - DNA, 23 bp, Artificial Sequence
  PRIMER
tccacctatctgcagtcttggac

SEQ ID NO: 45 - DNA, 23 bp, Artificial Sequence
  PRIMER
cctctgcttagtcagacaggaac

SEQ ID NO: 46 - DNA, 23 bp, Artificial Sequence
  PRIMER
ggactctactcggagtccaaatc

SEQ ID NO: 47 - DNA, 23 bp, Artificial Sequence
  PRIMER
tgctgcaggagtgaactttggtc

SEQ ID NO: 48 - DNA, 22 bp, Artificial Sequence
  PRIMER
cagggccacagctgcccagttc

SEQ ID NO: 49 - DNA, 22 bp, Artificial Sequence
  PRIMER
gtgcagggctgagctgataagc

SEQ ID NO: 50 - DNA, 23 bp, Artificial Sequence
  PRIMER
atggttcccgtgtctggaggcag

SEQ ID NO: 51 - DNA, 24 bp, Artificial Sequence
  PRIMER
actagggcaacactgagggttcac

SEQ ID NO: 52 - DNA, 23 bp, Artificial Sequence
  PRIMER
tatgcagtggactgtggagtgtg

SEQ ID NO: 53 - DNA, 23 bp, Artificial Sequence
  PRIMER
ggccacagagaggctgcactcca

SEQ ID NO: 54 - DNA, 22 bp, Artificial Sequence
  PRIMER
cctgaaggcccagggtctacga

SEQ ID NO: 55 - DNA, 22 bp, Artificial Sequence
  PRIMER
taagccaccagtggcatcttgc

SEQ ID NO: 56 - DNA, 22 bp, Artificial Sequence
  PRIMER
tttgtgtccacacccaagacgc

SEQ ID NO: 57 - DNA, 22 bp, Artificial Sequence
  PRIMER
tcaagcgtccttaggatggtgc

SEQ ID NO: 58 - DNA, 22 bp, Artificial Sequence
  PRIMER
atggtacgagactgggtgcttc

(19) United States
(12) Patent Application Publication
(10) Pub. No.: US 2011/0092796 A1
(43) Pub. Date: Apr. 21, 2011

(54) METHOD FOR DIAGNOSING CMT1A AND CMT2A BY MRI

(75) Inventors: Byung-Ok Choi, Gangnam-gu (KR); Ki-Wha Chung, Sungnam City (KR)

(73) Assignees: EWHA UNIVERSITY-INDUSTRY COLLABORATION FOUNDATION, Seoul (KR); KONGJU NATIONAL UNIVERSITY INDUSTRY ACADEMIA, Cheonan-si (KR)

(21) Appl. No.: 12/581,641

(22) Filed: Oct. 19, 2009

(51) Int. Cl. A61B 5/055

(52) U.S. Cl. 600/410

(57) ABSTRACT

Disclosed is a method for diagnosing Charcot-Marie-Tooth (CMT) disease. More specifically, disclosed is a method for diagnosing a subtype of Charcot-Marie-Tooth disease type 1, (i.e., CMT1A) and a subtype of the disease type 2 (i.e., CMT2A) by evaluating fatty infiltration behaviors in respective compartments of proximal lower extremity muscles via comparison and analysis of MRI on the proximal lower extremities. Further disclosed is a method for diagnosing CMT1A and CMT2A, by analyzing fatty infiltration levels between respective compartments by MRI examination on distal lower extremity muscles.

### BACKGROUND

1. Field

The present embodiments relate to diagnosis of Charcot-Marie-Tooth disease. More specifically, some embodiments relate to a method for diagnosing Charcot-Marie-Tooth Type 1 (CMT1A) and Charcot-Marie-Tooth Type 2 (CMT2A) by MRI examination on proximal lower extremities.

2. Description of the Related Art

In accordance with rapid progress in molecular genetics and bioinformatics, a great deal of research is underway to identify pathogenic genes and mechanisms of inherited disorders all over the world. Since the nature of inherited disorders was first established, the ideas relating to diagnosis and treatment of diseases have varied. One representative inherited disorder is Charcot-Marie-Tooth disease (hereinafter, simply referred to as “CMT”). CMT is an acronym of the names of the three physicians—Charcot and Marie (France), and Tooth (England), who first reported it in 1886. CMT is one of the most common inherited neurological disorders, affecting approximately 1 in 2,500 people. It has been regarded as a disorder in which the lower legs take on an “inverted champagne bottle” appearance due to atrophy of lower limb muscles, but is recently regarded as a group including genetically, clinically and electrophysiologically different diseases (Harding A E, Thomas P K. The clinical features of hereditary motor and sensory neuropathy types I and II. Brain 1980; 103:259-280.). CMT is difficult to diagnose due to its clinical and genetic diversity. Symptoms of CMT include weakness and loss of mobility in foot and hand muscles. As the disease progresses, difficulty in walking is increased or mobility aids such as wheelchairs may be required in early childhood.

A large amount of new information on pathogenic genes and mechanisms of CMT has been uncovered, and known pathogenic genes have reached 25 or more (Krajewski K M, Shy M E. Genetic testing in neuromuscular disease. Neurol Clin 2004; 22:481-508; Niemann A, Berger P, Suter U. Pathomechanisms of mutant proteins in Charcot-Marie-Tooth disease. Neuromolecular Med 2006; 8:217-241). A clear understanding of molecular-genetic mechanisms with regard to issues such as metabolic dysfunction of peripheral nerves, myelination, and correlations between myelin sheaths, axons and muscles, is important for treatment of the disease, which considerably contributes to pathophysiologic studies of CMT and complicated classification of phenotype and genotype thereof (Berger P, Young P, Suter U. Molecular cell biology of Charcot-Marie-Tooth disease. Neurogenetics 2002; 4:1-15). Recent animal test results ascertained its possibility. In particular, since onapristone, ascorbic acid and neurotrophin-3 (NT-3) useful as CMT1A disease drugs were reported, much attention has been focused on treatment as well as diagnosis. As a consequence, CMT1A can be inhibited by accurate genetic diagnosis and can be moderately treated depending on genetic causes (Sereda M W, Meyer zu Horste G, Suter U, Uzma N, Nave K A. Therapeutic administration of progesterone antagonist in a model of Charcot-Marie-Tooth disease (CMT-1A). Nat Med 2003; 9:1533-1537; Passage E, Norreel J C, Noack-Fraissignes P, Sanguedolce V, Pizant J, Thirion X, et al. Ascorbic acid treatment corrects the phenotype of a mouse model of Charcot-Marie-Tooth disease. Nat Med 2004; 10:396-401; Sahenk Z, Nagaraja H N, McCracken B S, King W M, Freimer M L, Cedarbaum J M, et al. NT-3 promotes nerve regeneration and sensory improvement in CMT1A mouse models and in patients. Neurology 2005; 65:681-689).

CMT which shows autosomal dominant inheritance is classified as CMT type 1 (CMT1) and CMT type 2 (CMT2), depending on whether primary pathogenesis is based on the myelin sheath or the axon (Harding and Thomas, 1980). CMT1A is caused by duplication of chromosomes 17p11.2-p12 including peripheral myelin protein 22 (PMP 22) genes and is the most common CMT1 subtype. CMT2A caused by mutations in 1VfFN2 is the most common CMT2 subtype (Jani-Acsadi A, Krajewski K, Shy M E. Charcot-Marie-Tooth neuropathies: diagnosis and management. Semin Neurol 2008; 28:185-194). CMT1A accounts for 70% of CMT1, and CMT2A accounts for 33% of CMT2 (Szigeti K, Garcia C A, Lupski J R. Charcot-Marie-Tooth disease and related hereditary polyneuropathies: molecular diagnostics determine aspects of medical management. Genet Med 2006; 8:86-92; Verhoeven K, Claeys K G, Zuchner S, Schroder J M, Weis J, Ceuterick C, et al. MFN2 mutation distribution and genotype/phenotype correlation in Charcot-Marie-Tooth type 2. Brain 2006; 129:2093-2102).

Those who suffer from CMT1A grow normally during childhood, express clinical symptoms before 20 years of age and then undergo gradual progression in symptoms (Harding and Thomas, 1980). CMT1A is associated with the PMP-22 gene on short arm of chromosome 17 (Lupski J R, de Oca-Luna R M, Slaugenhaupt S, Pentao L, Guzzetta V, Trask B J, et al. DNA duplication associated with Charcot-Marie-Tooth disease type 1A. Cell 1991; 66: 219-232). The subtype CMT1A is caused by unequal crossing-over of 1.4 Mb base pairs known as CMT1A-REP during meiosis, leading to duplication of chromosomes (Timmerman V, Nelis E, Van Hul W, Nieuwenhuijsen B W, Chen K L, Wang S, et al. The peripheral myelin protein gene PMP-22 is contained within the Charcot-Marie-Tooth disease type 1A duplication. Nat Genet 1992; 1:171-175). Also, the subtype CMT1A may be caused by a point mutation of PMP22 genes (Valentijn L J, Baas F, Wolterman R A, Hoogendijk J E, van den Bosch N H, Zorn I, et al. Identical point mutations of PMP-22 in Trembler-J mouse and Charcot-Marie-Tooth disease type 1A. Nat Genet 1992; 2:288-291). The onset of the disease completely conforms to Mendel's law and is directly associated with genetic defects. Accordingly, genetic test results confirm the accurate diagnosis rather than suggest any simple tendency or potentiality of disease-onset. (Vallat J M, Sindou P, Preux P M, Tabaraud F, Milor A M, Couratier P, et al. Ultrastructural PMP22 expression in inherited demyelinating neuropathies. Ann Neurol 1996; 39:813-817). The pathogenesis in which an excess amount of PMP22 causes CMT1A has not yet been accurately revealed. However, it was reported that myelin sheath abnormalities result in inhibition of interactions between the myelin sheath and the axon, causing damage to the axon and clinical symptoms due to dysfunction (Suter U, Scherer S S. Disease mechanisms in inherited neuropathies. Nat Rev Neurosci 2003; 4:714-726).

When compared to CMT1, CMT2 involves a variety of severity and onset-age (Bienfait H M, Baas F, Koelman J H, de Haan R J, van Engelen B G, Gabreels-Festen A A, et al. Phenotype of Charcot-Marie-Tooth disease Type 2. Neurology 2007; 68:1658-1667). Further, CMT1 decreases nerve conduction velocity, whereas CMT2 normal or slightly decreases nerve conduction velocity (Harding and Thomas, 1980). CMT2A patients were reported to have two different clinical behaviors, i.e., early-onset severe group, wherein the disease arises early (prior to 10 years old) and shows severe symptoms, and late-onset mild group, wherein the disease arises late (after 10 years old) and shows light symptoms (Chung K W, Kim S B, Park K D, Choi K G, Lee J H, Eun H W, et al. Early onset severe and late-onset mild Charcot-Marie-Tooth disease with mitofusin 2 (mfn2) mutations. Brain 2006; 129:2103-2118; Verhoeven K, Claeys K G, Zuchner S, Schroder J M, Weis J, Ceuterick C, et al. MFN2 mutation distribution and genotype/phenotype correlation in Charcot-Marie-Tooth type 2. Brain 2006; 129:2093-2102). CMT2A-causing genes were first reported by Zuchner et al. in 2004 to be MFN2 genes, which greatly affect mitochondrial functions, and a great deal of research associated therewith has been continuously reported (Zuchner S, Mersiyanova I V, Muglia M, Bissar-Tadmouri N, Rochelle J, Dadali E L, et al. Mutations in the mitochondrial GTPase mitofusin 2 cause Charcot-Marie-Tooth neuropathy type 2A. Nat Genet 2004; 36:449-451; Reilly M M. Axonal Charcot-Marie-Tooth disease: The fog is slowly lifting Neurology 2005; 65:186-187). Mutations in MFN2 genes cause the mitochondria to fail to move towards the axon in nerve cells, causing dysfunction of the mitochondria, dysfunction of peripheral nervous systems and expression of clinical symptoms of CMT2A (Chen H, Detmer S A, Ewald A J, Griffin E E, Fraser S E, Chan D C. Mitofusins Mfn1 and Mfn2 coordinately regulate mitochondrial fusion and are essential for embryonic development. J Cell Biol 2003; 160:189-200). Disruption of mitochondrial fusion into the axon is reported to be due to dysfunction of coupling of mitochondria and kinesin, which is required for transport in the axon (Chen H, Chomyn A, Chan D C. Disruption of fusion results in mitochondrial heterogeneity and dysfunction. J Biol Chem 2005; 280:26185-26192).

MRI plays an important role in evaluation of pathological conditions of skeletal muscles based upon variation of intensity in muscle signals, and variation in MR signals occurs due to increased extracellular fluid proportions or varied moisture distribution (Fleckenstein J L, Watumull D, Conner K E, Ezaki M, Greenlee R G Jr., Bryan W W, et al. Denervated human skeletal muscle: MR imaging evaluation. Radiology 1993; 187:213-218; May D A, Disler D G, Jones E A, Balkissoon A A, Manaster B J. Abnormal signal intensity in skeletal muscle at MR imaging: patterns, pearls, and pitfalls. Radiographics 2000; 20:5295-315; Farber J M, Buckwalter K A. MR imaging in nonneoplastic muscle disorders of the lower extremity. Radiol Clin North Am 2002; 40:1013-1031). The characteristic findings indicate that muscle atrophy due to nervous injury shows high signal intensities at T1-weighted spin echo imaging. Accordingly, MRI of the lower extremities to evaluate muscular variations including secondary muscle atrophy and fat infiltration which are caused by nervous system injuries such as demyelination or axon injury is known to be suitable for use in evaluating and analyzing neuropathies (Polak J F, Jolesz F A, Adams D F. Magnetic resonance imaging of skeletal muscle. Prolongation of T1 and T2 subsequent to denervation. Invest Radiol 1988; 23:365-369). As a result of computed tomography (CT) analysis and comparison of the distal lower extremity, with respect to the overall CMP patients diagnosed, based on only clinical behaviors without classifying subtypes using gene mutation testing, the presence of two types, i.e., a peroneal nerve type (P-type) wherein peroneal-innervated muscles are first injured, and a tibial nerve type (T-type) wherein tibial nerve innervated muscles are dominantly injured was reported (Price et al., 1993). It was also reported that the P-type suitably conforms to length-dependent neuropathy theory, while the T-type does not conform to the general characteristics of a neuropathy (Price A E, Maisel R, Drennan J C. Computed tomographic analysis of pes cavus. J Pediatr Orthop 1993; 13:646-653). Gallardo et al. performed MRI on the distal lower extremities of patients suffering from demyelinating neuropathy, CMT1A, and revealed that the patients underwent only P-type injuries (Gallardo E, Garcia A, Combarros O, Berciano J. Charcot-Marie-Tooth disease type 1A duplication: Spectrum of clinical and magnetic resonance imaging features in leg and foot muscles. Brain 2006; 129:426-437).

### SUMMARY

The present embodiments generally relate to diagnosis of Charcot-Marie-Tooth disease. More specifically, some embodiments relate to a method for diagnosing Charcot-Marie-Tooth Type 1 (CMT1A) and Charcot-Marie-Tooth Type 2 (CMT2A) by MRI examination on proximal lower extremities.

The inventors of the present embodiments compared and analyzed the difference in distal lower extremity MRI features between demyelinating neuropathy (CMT1A) patients and axonal neuropathy (CMT2A) patients. The findings indicate that CMT1A patients are P-type, whereas CMT2A patients are partially T-type wherein Gastrocnemius is mainly injured, which reveals that CMT1A and CMT2A patients show different fatty inflation patterns (Chung K W, Suh B C, Shy M E, Cho S Y, Yoo J H, Park S W, et al. Different clinical and magnetic resonance imaging features between Charcot-Marie-Tooth disease type 1A and 2A. Neuromuscul Disord 2008; 18:610-618; which is incorporated herein by reference in its entirety).

However, to date, a great deal of research on CMT patients has been based on the assumption that distal muscle injuries are dominantly expressed in CMT patients and proximal muscle injuries are hardly expressed therein. For this reason, there is no comparison and analysis of MRI performed on the proximal lower extremities in CMT1A or CMT2A patients. Accordingly, the inventors of the present technology surprisingly discovered the differences between CMT and CMT2A via MRI on proximal lower extremities in CMT and CMT2A patients. Based on these findings, a diagnosis method finally has been completed.

Therefore, embodiments relate generally to methods for diagnosing Charcot-Marie-Tooth (CMT) disease, and more specifically, some embodiments relate to a method for diagnosing a subtype of Charcot-Marie-Tooth disease type 1, (i.e., CMT1A) and a subtype of the disease type 2 (i.e., CMT2A) via comparison and analysis of MRI on proximal lower extremities.

In one aspect a method for diagnosing Charcot-Marie-Tooth disease (CMT) may include confirming a fatty infiltration increase in one or more compartments of proximal lower extremities by MRI examination of the proximal lower extremities of a patient. In some aspects, it can be seen from MRI examination of the proximal lower extremities that even symptom-free patients as CMT patients that are normal in thigh muscle MRC showed fatty infiltration in at least one of anterior compartments, medial compartments and posterior compartments. The diagnosis of the CMT may be carried out by confirming fatty infiltration in one or more compartments on proximal lower extremities by MRI examination of the proximal lower extremities.

In accordance with another aspect, provided is a method for diagnosing CMT1A or CMT2A, comprising comparing a fatty infiltration level between anterior compartments, medial compartments and posterior compartments by MRI examination of proximal lower extremity muscles of a patient.

More specifically, the diagnosis of the CMT1A may include, for example, confirming the behavior that fatty infiltration severity in the proximal lower extremity muscle increases in this order: anterior compartments, medial compartments, and posterior compartments.

The diagnosis of the CMT2A may include, for example, confirming the behavior that the fatty infiltration severity in the proximal lower extremity muscles increases in this order: medial compartments, posterior compartments and anterior compartments.

The analysis results of fatty infiltration behaviors in proximal lower extremity muscles for CMT1A and CMT2A patients via MRI examination on the proximal lower extremities indicate that CMT1A increases in fatty infiltration severity in this order: anterior compartments<medial compartments<posterior compartments, whereas CMT2A increases in fatty infiltration severity in this order: medial compartments<posterior compartments<anterior compartments. This indicates that the two diseases are clearly distinguished from each other in fatty infiltration behaviors on proximal lower extremity muscles. Accordingly, some embodiments provide a method for diagnosing CMT1A or CMT2A by comparing a fatty infiltration level between proximal lower extremity muscle compartments by MRI examination on proximal lower extremity muscles.

Also, some embodiments provide a method for distinguishing CMT1A from CMT2A by comparing a fatty infiltration level between one or more of anterior compartments, medial compartments and posterior compartments by MRI examination of proximal lower extremity muscles. In the case where clinical symptoms confirmed that patients were affected by CMT, but whether the CMT is CMT1A or CMT2A is not clear, CMT1A and CMT2A can be clearly distinguished from each other by comparing fatty infiltration in one or more of anterior, medial and posterior compartments of proximal lower extremity muscles via MRI examination of proximal lower extremities.

### BRIEF DESCRIPTION OF THE DRAWINGS

The objects, features and other advantages of the embodiments will be more clearly understood from the following detailed description taken in conjunction with the accompanying drawings, in which:

FIG. 1 is images illustrating compartment analysis of thigh muscles. More specifically, (A) is an image showing the following respective thigh muscles: vastus lateralis (VL); vastus intermedius (VI); vastus medialis (VM); rectus femoris (RF); sartorius (Sr); adductor magnus (AM); gracilis (Gr); biceps femoris (BF); semitendinosus (ST); and semimembranosus (SM). (B) Three compartments, i.e., anterior, posterior and medial compartments constitute the thigh region. Green anterior compartments include quadriceps musculature consisting of RF, VM, VL and VI. Yellow posterior compartments include hamstring muscles consisting of SM, ST and BF. Gr and AM are present in violet medial compartments.

FIG. 2 is a graph showing fatty infiltration in anterior, medial and posterior compartments of proximal lower extremities for CMT1A and CMT2A patients. Muscular fatty infiltration severity of CMT2A is increased in all three compartments, as compared to CMT1A. CMT2A is more selectively correlated with anterior compartments. CMT1A shows fatty infiltration in thigh posterior medial compartments.

FIG. 3 is a graph showing correlation between fatty infiltration scores on thigh muscles and MRC lower extremity muscle strength grades (r; correlation coefficient). (A) is a graph showing good correlation between weak strength of knee-extension muscles and high fatty infiltration in anterior compartments of thigh muscles. (B) is a graph showing strong correlation between weak knee-flexion muscles and high fatty infiltration in posterior compartments of thigh muscles.

FIG. 4 is a series of images showing sequential muscle association behaviors with increase in the duration of disease for CMT1A patients (A-C) and CMT2A patients (D-F). (A) A 38 year old female patient who had suffered from CMT1A disease for 11 years showed partial fatty infiltration in posterior compartments. (B) A 63 year old female patient who had suffered from CMT1A disease for 24 years (FIG. 4B) showed considerable fatty infiltration in posterior compartments of thigh muscles (C) A 86 year old female patient who had suffered from the disease for 31 years showed more severe fatty infiltration in both posterior and medial compartments (D) A 14 year old male patient who had suffered from the CMT2A disease for 10 years showed medium fatty infiltration in anterior compartments, and slight fatty infiltration in posterior compartments (E) A 36 year old female patient who had suffered from the CMT2A disease for 28 years showed severe fatty infiltration in anterior compartments and medium fatty infiltration in posterior compartments (F) A 40 year old female patient who suffer from the CMT2A disease for 31 years showed severe fatty infiltration in all compartments except a part of medial compartments.

FIG. 5 shows MRI results of CMT1A with demyelinating neuropathy. (A-D) MRI results obtained from lower extremities including the foot, leg and thigh correspond to length-dependent injury theory. Fatty infiltration severity gradually increases towards the distal. (E-F) Fatty infiltration in the thigh and leg muscles on lower extremities shows strong correlation between severity and distance.

FIG. 6 shows MRI results of axonal neuropathy, CMT2A. (A-D) MRI results obtained from lower extremities including the foot, leg and thigh correspond to length-dependent injury theory. (E) However, on proximal lower extremities, axonal CMT showed increased fatty infiltration severity in anterior compartments muscles, as compared to anterior compartments of demyelinating neuropathy, CMT1A. (F) In addition, Gastrocnemius is the most rapid and severe of other leg muscles.

### DETAILED DESCRIPTION OF THE PREFERRED EMBODIMENTS

Hereinafter, the present embodiments are described in more detail with reference to the following non-limiting Examples including specific examinations and analysis examples.

### 1. Research Subject and Method

(1) Subject

This research was tested for 37 patients (15 males, 22 females) who were given a diagnosis of CMT based on neurological diagnostic findings and electrophysiological inspection and visited the department of neurology, or were requested from other sources, and underwent MRI on proximal lower extremities due to accurate diagnosis of PMP22 or MFN2 gene mutations. Of the patients, the number of CMT1A patients showing autosomal dominant inheritance and demyelinating neuropathy was 26, and the number of CMT2A showing autosomal dominant inheritance and axonal neuropathy was 11. A normal control group with respect to gene mutations included 105 individuals (43 males, 62 females) who were free of neuropathic findings in clinical and neurological examination and nerve conduction tests, as well as family history of CMT. All patients, parents of minors and the normal control group participated in an interview associated with contents of the study, were fully informed of the disease and consented to genetic testing. A written consent and consent procedure were obtained in accordance with regulations of the medical ethics committee, Ewha Medical Center.

(2) Genetic Testing

Peripheral blood was collected from a test group (CMT types of patients and their families) and a normal control group in an EDTA-treated tube, genomic DNA was isolated from whole blood using QIAamp DNA blood kits (Qiagen, Hilden, Germany). PMP22 gene testing was carried out by observing locus duplication using six microsatellite markers (i.e., D17S921, D17S9B, D17S9A, D17S918, D17S4A and D17S122) showing short tandem repeats (STR), located on the chromosome 17p11.2-12 in the genomic DNA of patients and their families. The primers for PCR on respective genes are as follows (Table 1). A multiplex system I was HEX-marked with D17S9B, D17S9A and D17S918, and a multiplex system II was FAM-marked with D17S921, D17S4A and D17S122. PCR amplification was carried out in a total of 20 μl reaction volume containing 10-20 ng DNA, different concentrations of primer solutions, 200 μM dNTPs, 2 mM MgCl₂, and 0.6 unit Tag DNA polymerase in a GeneAmp PCR system 2700 (Applied Biosystems, USA). The PCR was carried out by pre-denaturing at 96° C. for 3 minutes, denaturing at 94° C. for one minute, annealing at 60° C. for 30 minutes, extension (elongation) at 70° C. for 90 seconds, repeating the series of previous steps 32 times and final-extension at 60° C. for 45 minutes. In addition, to observe point mutations, PMP22 genes were amplified on the exons and adjacent introns by PCR and base sequences thereof were then analyzed, to search for specific mutations (Table 2).

TABLE 1
Microsatellite markers and PCR conditions
for detection of 17p11.2-12 duplication

| | | | Concentration |
|---|---|---|---|
| | Sequence of primers | Dye | of primer |
| Locus | (5′ -> 3′) | label | (uM) |
| D17S921 | F: GTGTTGTATTAG | FAM | 0.06 |
| | GCAGAGTTCTCC | | |
| | (SEQ ID NO: 1) | | |
| | R: GGCAGTAGATGG | | |
| | TGACTTTATGGC | | |
| | (SEQ ID NO: 2) | | |
| D17S9B | F: TCTCAGTCCTGA | HEX | 0.50 |
| | TTTCTTGATTTTG | | |
| | (SEQ ID NO: 3) | | |
| | R: CCAGAGCTAACA | | |
| | CCACATTCA | | |
| | (SEQ ID NO: 4) | | |
| D17S9A | F: CAACCATCAGTGA | HEX | 1.00 |
| | TTTGATGGTTTAC | | |
| | (SEQ ID NO: 5) | | |
| | R: GAGTTGTCACTAG | | |
| | AACCCTGTTC | | |
| | (SEQ ID NO: 6) | | |
| D175918 | F: TCCTGTAATCTGT | HEX | 0.25 |
| | CCCCAAACGTC | | |
| | (SEQ ID NO: 7) | | |
| | R: TTCCTCACACAAC | | |
| | CTATTGATAGTC | | |
| | (SEQ ID NO: 8) | | |
| D17S4A | F: CTGTGGAGGAAAGA | FAM | 0.20 |
| | AAACACTGCC | | |
| | (SEQ ID NO: 9) | | |
| | R: GCACTAAAGTAGCT | | |
| | TGTAACTCTG | | |
| | (SEQ ID NO: 10) | | |
| D1752230 | F: AGGAAACTGATGTC | FAM | 0.20 |
| | TAAAACTATCC | | |
| | (SEQ ID NO: 11) | | |
| | R: GTGAATCCAGGAGG | | |
| | CAGAGCTTGC | | |
| | (SEQ ID NO: 12) | | |

TABLE 2
Primer sequences for PCR
amplification of PMP22 exons

| Exon | Primer sequence (5′ -> 3′) |
|---|---|
| Promoter | F: CACAGATGCATGGGATAGGGGTCC |
| | (SEQ ID NO: 13) |
| | R: ATGGAGATGAGGTACAGTGTGGGC |
| | (SEQ ID NO: 14) |
| Exon 1A | F: ATCTCTGCAGAATTCACTGGGAG |
| | (SEQ ID NO: 15) |
| | R: GCATAGGCACACATCACCCAGAG |
| | (SEQ ID NO: 16) |
| Exon 1B | F: TCGTAGGTGCAGACAGTAATAGC |
| | (SEQ ID NO: 17) |
| | R: ATGCCCACATTTCTCCTCCATCTC |
| | (SEQ ID NO: 18) |
| Exon 2 | F: CGCAGGCAGAAACTCCGCTGAGC |
| | (SEQ ID NO: 19) |
| | R: CGTCTGAGACTTCCAACTGTCTGC |
| | (SEQ ID NO: 20) |
| Exon 3 | F: AACGTTGGCTCTTACCATGCAGG |
| | (SEQ ID NO: 21) |
| | R: TGGACTCATGGCTCCCTGTCAC |
| | (SEQ ID NO: 22) |
| Exon 4 | F: ATGTATGTAATGTGTACATATTGGCAC |
| | (SEQ ID NO: 23) |
| | R: TTGGATGCACCCCGCTTCCACA |
| | (SEQ ID NO: 24) |
| Exon 5 | F: TCTGCCATGGACTCTCCGTCAC |
| | (SEQ ID NO: 25) |
| | R: GTTTGGGATTTTGGGCTAGCTC |
| | (SEQ ID NO: 26) |

Mutations were searched for by sequencing the exons and adjacent introns encoding GTPase domains and their peripheral sites in MFN2 genes (Table 3). The exons 7-8 and 10-11 were simultaneously amplified as one fragment and the remaining exons were separately amplified. The PCR reaction to amplify the corresponding sites of MFN2 genes was carried out by pre-denaturing in a 50 μl reaction solution containing template DNA 30-50 ng, each primer 10 pmol, dNTPs 200 μM, MgCl₂ 1.5 mM, Taq polymerase 0.5 U and 1× reaction buffer (Promega, Madison, Wis., USA) in a PCR amplification system (ABI GeneAmp 9700, Foster City, Calif., USA) at 94° C. for 2 minutes, 32-cycles, each including temperature steps: 94° C. for 40 seconds, 60° C. for 30 seconds and 72° C. for 90 seconds, and further elongation at 72° C. for 7 minutes. The DNA amplified by PCR was purified and its base sequences of both directions were determined in an automated sequencer (ABI 3700®, Foster City, Calif., USA) using a BigDye Terminator Cycle Sequencing Kit. CHROMAS (Ver. 2.23) program was used to read DNA sequences.

TABLE 3
Oligonucleotide primer sequences for
screening of MFN2 gene mutations

| Exon sites | Primer sequence (5′-> 3′) |
|---|---|
| Exon 1 | F: ATGATGCAGTGGGAGTCCGAGC |
| | (SEQ ID NO: 27) |
| | R: ACGCCGAGCTGCTCAGGACTCGC |
| | (SEQ ID NO: 28) |
| Exon 2 | F: AGCTTCCCTCAGGGATGAGTTTG |
| | (SEQ ID NO: 29) |
| | R: ACTCAGCTCAAATCCAGGGTGC |
| | (SEQ ID NO: 30) |
| Exon 3 | F: AGCATTCCGCCATCTCCCTAGCAG |
| | (SEQ ID NO: 31) |
| | R: CACCCGCAAATCCCACTAGCTAA |
| | (SEQ ID NO: 32) |
| Exon 4 | F: TCCAGACTTGGGACTGTGGAAC |
| | (SEQ ID NO: 33) |
| | R: TGGAACGTTCTGTGACCTTGCAC |
| | (SEQ ID NO: 34) |
| Exon 5 | F: CCAGGCTGGTATCTGAGTTGTGA |
| | (SEQ ID NO: 35) |
| | R: GTGTCACAACGGAGGACTTGCTC |
| | (SEQ ID NO: 36) |
| Exon 6 | F: TGTGATGCAGCGGCACAGGAAATC |
| | (SEQ ID NO: 37) |
| | R: TGGTGCCTTCCAGTTTGGACCTC |
| | (SEQ ID NO: 38) |
| Exon 7-8 | F: TAGGGVTCCTGCTCTGCCTGATGA |
| | (SEQ ID NO: 39) |
| | R: AGTGCTCCCTCGGGGTTGCATTC |
| | (SEQ ID NO: 40) |
| Exon 9 | F: GCCCAGCCTCTTATGACCTATTC |
| | (SEQ ID NO: 41) |
| | R: TGACAGACTCCTCAGCACGAGAC |
| | (SEQ ID NO: 42) |
| Exon 10-11 | F: CTGCTGCCAAGTTGTTTCTGGAC |
| | (SEQ ID NO: 43) |
| | R: TCCACCTATCTGCAGTCTTGGAC |
| | (SEQ ID NO: 44) |
| Exon 12 | F: CCTCTGCTTAGTCAGACAGGAAC |
| | (SEQ ID NO: 45) |
| | R: GGACTCTACTCGGAGTCCAAATC |
| | (SEQ ID NO: 46) |
| Exon 13-14 | F: TGCTGCAGGAGTGAACTTTGGTC |
| | (SEQ ID NO: 47) |
| | R: CAGGGCCACAGCTGCCCAGTTC |
| | (SEQ ID NO: 48) |
| Exon 15 | F: GTGCAGGGCTGAGCTGATAAGC |
| | (SEQ ID NO: 49) |
| | R: ATGGTTCCCGTGTCTGGAGGCAG |
| | (SEQ ID NO: 50) |
| Exon 16 | F: ACTAGGGCAACACTGAGGGTTCAC |
| | (SEQ ID NO: 51) |
| | R: TATGCAGTGGACTGTGGAGTGTG |
| | (SEQ ID NO: 52) |
| Exon 17 | F: GGCCACAGAGAGGCTGCACTCCA |
| | (SEQ ID NO: 53) |
| | R: CCTGAAGGCCCAGGGTCTACGA |
| | (SEQ ID NO: 54) |
| Exon 18 | F: TAAGCCACCAGTGGCATCTTGC |
| | (SEQ ID NO: 55) |
| | R: TTTGTGTCCACACCCAAGACGC |
| | (SEQ ID NO: 56) |
| Exon 19 | F: TCAAGCGTCCTTAGGATGGTGC |
| | (SEQ ID NO: 57) |
| | R: ATGGTACGAGACTGGGTGCTTC |
| | (SEQ ID NO: 58) |

In the case where a mutation detected by base sequence analysis is observed only in the corresponding family of patients, is not observed in members of the non-patient group and is not observed in any of the 105 randomized normal individuals, the mutation is defined as a causal mutation.

(3) Clinical Evaluation

Onset age, disease duration, functional disability scale (FDS) to indicate disability by a disease, muscle atrophy, foot deformities and scoliosis were investigated to compare clinical behaviors of CMT. In addition, disorders of heel- or toe-walking and strength of thigh muscles in different sites were investigated, and the examination was video-recorded. The onset age is defined as the time at which symptoms of CMT such as deterioration in motor and sensory sensations or foot deformities first occur. The disease duration is defined as a period which is taken from a hospital-visit day to the onset age.

The strength evaluation in thigh muscles was carried out based on the diagnosis results obtained by three neurologists (Choi, Kim and Kim) using standardized medical research council (MRC) which is the most widely manual testing scale, graded as follows: 0=no muscular contraction; 1=slight muscular contraction; 2=the ability to move in the absence of gravity; 3=the ability to contract against gravity, but not against some additional resistance; 4=the ability to move the joint against combination of gravity and some resistance, but not normal; and 5=normal. CMT disease severity was assessed according to a nine-point functional disability score (FDS): 0=normal; 1=normal, but with cramps and fatigability; 2=inability to run; 3=walking difficult but still possible unaided; 4=able to walk with a cane; 5=able to walk with crutches; 6=able to walk with a walker; 7=wheelchair-bound; and 8=bedridden (Birouk et al., 1997).

(4) Electrophysiological Testing

Median and lunar nerves as motor and sensory nerves in the upper extremities, and common peroneal nerves and posterior tibial nerves as motor nerves and sural nerves as sensory nerves in the lower extremities were tested in different compartments for all test subjects. The testing was carried out by skin surface stimulation and recording techniques using Cadwell 5200, Dantec 1500 and Synergy™ (Medelec) in accordance with the method reported by Oh (Oh S J. Clinical Electromyography: nerve conduction studies. 2nd ed. Baltimore: Williams & Wilkins, 1993:37-53; which is incorporated herein by reference in its entirety). For the motor nerves, terminal latency (TL), compound muscle action potential (CMAP) amplitude and nerve conduction velocity (NCV) in different compartments and for the sensory nerves, nerve conduction velocity (NCV) and compound nerve action potential (CNAP) amplitude were measured.

TL is obtained in msec by measuring an exit zone of a CMAP after motor nerve stimulation. The motor and sensory nerve conduction velocities were obtained in msec. Potential amplitude was obtained by measuring the distance from a positive maximum (positive peak) to a negative maximum (negative peak), wherein sensory nerves are represented by microvolts (μV) and motor nerves are represented by millivolts (mV). The nerve conduction tests were determined based on the standards suggested by the hospital's electromyography lab. Motor nerve conduction velocity was regarded to be normal, when median nerves ≧50.5 m/s, ulnar nerves ≧51.1 m/s, common peroneal nerves ≧40.5 m/s, and posterior tibial nerves ≧41.1 m/s. Sensory nerve conduction velocity was regarded to be normal, when median nerves ≧39.3 m/s, ulnar nerves ≧37.5 m/s and sural nerves ≧32.1 m/s. The compound muscle action potential was regarded to be normal, when median nerves ≧6.0 mV, ulnar nerves ≧8.0 mV, common peroneal nerves ≧1.5 mV, and posterior tibial nerves ≧6 mV. The compound nerve action potential was regarded to be normal when median nerves ≧8.4 μV, ulnar nerves ≧7.9 μV, and sural nerves ≧6.0 μV.

(5) Imaging Test

MRI was performed on the lower extremities including the thigh in 37 patients. The MRI examination was carried out in 1.5T system (Siemens Vision; Siemens, Erlangen, Germany). MRI examination was carried out in all patients using a phase-array multicoil such that the bilateral lower extremities were arranged along the center of the coil and were simultaneously taken, while the patients lay flat on their back. In order to clearly define a boundary of different compartments of images for the thigh and leg muscles in the lower extremities, images of axial planes (field of view; 24-32 cm, slice thickness; 10 mm, slice gap; 0.5-1.0 mm) and coronal planes (field of view; 38-40 cm, slice thickness; 4-5 mm, slice gap; 0.5-1.0 mm) were obtained. Images were obtained in both axial and coronal planes for all patients using T1-weighted fast spin-echo (TR/TE; 570-650/14-20, matrixes; 512), T2-weighted fast spin-echo (TR/TE 2800-4000/96-99, matrixes; 512), and fat-suppressed T2-weighted spin echo (TR/TE 3090-4900/85-99, matrix; 512) pulse sequences. The aforementioned protocol was commonly applied to the thighs, calves and feet in the lower extremities. In addition, enhance contrast images were obtained using fat-suppressed T1-weighted axial spin-echo (TR/TR 600/14, 512 matrixes) before and after intravenous injection of a paramagnetic contrast agent (Gd-DPTA; Magnevist, Schering, Germany 0.2 ml/kg).

(6) Assay of Cross-Section and Fatty Infiltration in Thigh Muscles

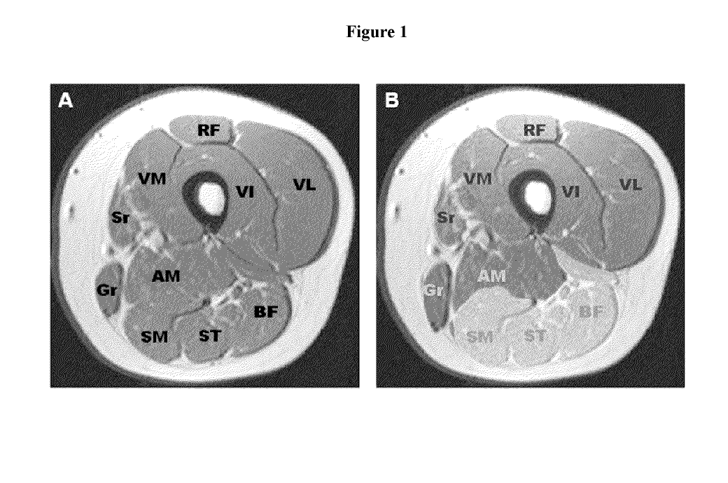

A length from the anterior greater trochanter to the flat lateral condyle on the distal knee joint was measured, and a cross-sectional area of the medial fascia lata, referred to a region of the MRI cross-section corresponding to the mid-thigh where there is no subcutaneous fat was measured. In proximal lower extremity MRI, anterior compartments include a group of vastus lateralis consisting of vastus intermedius (VL) vastus medialis (VM), rectus femoris (RF) and sartorius (Sr), wherein thigh nerves are dominant, and medial compartment include adductor magnus (AM), adductor longus (AL) and gracilis (Gr) wherein obturator nerves are dominant, and posterior compartments include biceps femoris (BF), semitendinosus (ST) and semimembranosus (SM) wherein sciatic nerves are dominant. These three compartments were compared with one another (FIG. 1; Anderson M W, Temple H T, Dussault R G, Kaplan P A. Compartmental anatomy: relevance to staging and biopsy of musculoskeletal tumors. AJR 1999; 173:1663-1671). Assessment of fatty infiltration shown on MRI was based on a five grading scale of 0-4: 0=no fat signal; 1=some fatty streaks, 2=fat occupying a minor part of muscle; 3=similar amount of fat and muscle; and 4=fat occupying the greater part of muscle) (Goutallie et al., 1994).

(7) Statistical Analysis

MRI findings were obtained by comparing the differences between disease groups due to gene mutations and analyzing correlations between clinical findings. The mean and standard deviation were compared using a test method such as independent sample t test, and Mann-Whitney U test. Variate spearman correlation was used to compare correlations between lower extremity muscle strength MRC grades, lower extremity MRI cross-sections and fatty infiltration. When p is less than 0.05, the result was regarded to be statistically significant. The statistical analysis was performed using SPSS for Windows®, Ver. 12.0 (SPSS Inc., Chicago, Ill.).

### 2. Results and Analysis

(1) Genetic Testing

The results of the genetic testing obtained by proximal lower extremity MRI in 37 patients are as follows. 26 CMT1A patients discovered to have demyelinating neuropathy all had PMP22 gene mutations, 23 patients showed duplication mutations of PMP22 genes, one showed partial duplication mutations, and two showed point mutations on Thr23Arg. 11 CMT2A patients showing axonal neuropathy showed six MFN2 gene mutations. These mutations were located in GTPase domains or adjacent thereto in the fourth (Leu92Pro, Arg94Trp), sixth (His165Arg), ninth (Arg280H is) and eleventh (Ser350Pro, Arg364Trp) exons.

These subjects were tested for MPZ, GDAP1, GJB1, EGR2, NEFL, DNM2, ALS4, ARHGEF10, DCTN1, GARS, LMNA, SEPT9, RAB7, LITAF, PRX, PARS, PRPS1, HSP22, HSP27 and BSCL2 known as CMT-causing mutant genes. The results indicate that no gene mutation was observed.

(2) Clinical Characteristics

Clinical characteristics are shown in Table 4. The CMT1A patient group comprised a total of 26 patients (10 males, 16 females) whose average age at the time of examination was 38.5 years old (range; 8-86), whose average age at onset was 14.8 years old (range; 3-55), and whose average disease duration was 23.8 years (range; 3-67). On the other hand, the CMT2A patient group comprised a total of 11 patients (5 males, 6 females) whose average age at the time of examination was 32.1 years old (range; 9-67), whose average age at onset was 11.1 years old (range; 1-33), and whose average disease duration was 21.0 years (range; 4-34). As a result of comparison in clinical behaviors, there was no statistically significant difference between the CMT1A patients and CMT2A patients in gender ratio, age at the time of examination, and duration of disease. Symptom-free patients accounted for 12% of the CMT1A group, but were not observed in the CMT2A group. The CMT2A group showed significantly increased muscle atrophy, as compared to the CMT1A group, but showed muscle atrophy and weakness in lower extremities, comparable to the muscle atrophy. In sensory dysfunction and deep tendon reflex, there was no statistically significant difference between the two groups. In FDS, to assess disability, CMT2A group is significantly higher than the CMT1A group (p<0.01). In toe-walking disorders, the CMT2A group is significantly higher than the CMT1A group (p<0.01). On the other hand, in heel-walking disorders, there was no statistically significant difference between the two groups.

TABLE 4
Basic characteristics of CMT1A and CMT2A patients

| | CMT1A | CMT2A | P |
|---|---|---|---|
| Number of patients | 26 | 11 | |
| Female ratio | 62% | 55% | NS |
| Age at the time of examination | 38.5 ± 21.2 | 32.1 ± 16.6 | NS |
| Onset age | 14.8 ± 12.6 | 11.1 ± 9.3 | NS |
| Disease duration (years) | 23.8 ± 17.3 | 21.0 ± 9.9 | NS |
| Asymptomatic | 3 (12%) | 0 (0%) | NS |
| Muscular strength weakness | | | |
| Upper extremities | 16 (62%) | 11 (100%) | NS |
| Lower extremities | 19 (73%) | 11 (100%) | NS |
| Muscular atrophy | | | |
| Upper extremities | 12 (46%) | 10 (91%) | <0.05 |
| Lower extremities | 16 (62%) | 11 (100%) | NS |
| Sensory dysfunction | | | |
| Pain | 17 (65%) | 11 (100%) | NS |
| Proprioception | 20 (77%) | 11 (100%) | NS |
| Areflexia | | | |
| Upper extremities | 19 (73%) | 8 (73%) | NS |
| Lower extremities | 22 (85%) | 11 (100%) | NS |
| FDS | 2.0 ± 1.6 | 4.5 ± 2.0 | <0.01 |
| Toe-walking dysfunction | 6 (23%) | 10 (91%) | <0.01 |
| Heel-walking dysfunction | 20 (77%) | 8 (73%) | NS |
| Foot deformities | 25 (96%) | 11 (100%) | NS |

NS means that p-value on comparison between CMT1A and CMT2A values is not significant, and
FDS means functional disability scale

The results of MRC muscle strength tests in lower extremities are shown in Table 5. The results indicate that CMT2A patients had decreased muscle strength, as compared to CMT1A patients. In flexion and extension of the knee joint, the CMT2A group was greatly reduced in muscle strength, as compared to the CMT1A group. The analysis results of muscle strength weakness in proximal lower extremities indicate that the CMT2A group was significantly decreased, as compared to the CMT1A group, and more specifically, the CMT2A group showed a 73% decrease in muscle strength of knee-extension muscles and a 55% decrease in muscle strength of knee-flexion muscles.

TABLE 5
Decrease frequency and MRC grades of muscle strength
in proximal lower extremities of CMT1A and CMT2A patients

| | CMT1A | CMT2A | P |
|---|---|---|---|
| Number of patients | 26 | 11 | |
| MRC scale | | | |
| Hip adduction | | | |
| The right | 5.00 ± 0.00 | 4.79 ± 0.58 | NS |
| The left | 5.00 ± 0.00 | 4.79 ± 0.58 | NS |
| knee joint extension | | | |
| The right | 4.90 ± 0.25 | 3.71 ± 1.60 | <0.01 |
| The left | 4.90 ± 0.25 | 3.67 ± 1.61 | <0.01 |
| knee joint flexion | | | |
| The right | 4.90 ± 0.32 | 4.12 ± 1.42 | <0.05 |
| The left | 4.90 ± 0.32 | 4.13 ± 1.45 | <0.05 |
| Muscular strength weakness at | | | |
| proximal muscles | | | |
| Hip adduction | 0 (0%) | 2 (18%) | NS |
| Knee-extension muscles | 4 (15%) | 8 (73%) | <0.01 |
| Knee-flexion muscles | 3 (12%) | 6 (55%) | <0.05 |

NS means that p-value on comparison between CMT1A and CMT2A values is not significant; and
MRC means medical research council.

(3) Electrophysiological Properties

All of the 37 subjects who underwent MRI examination on proximal lower extremities participated in electrophysiological tests. Motor nerves were measured in median, lunar, common peroneal and posterior tibial nerves, and sensory nerves were measured in median, lunar and sural nerves. The results thus obtained are shown in Table 6. There was significant difference between the CMT1A and CMT2A groups in motor nerve conduction velocity and terminal latency in median nerves and lunar nerves, as well as in nerve conduction velocity. The MRI findings revealed that CMT1A has a motor nerve conduction velocity in median nerves of less than 38 m/s, which corresponds to terminal latency-delayed demyelinating neuropathy, and on the other hand, the CMT2A group developed axonal neuropathy.

TABLE 6
Motor and sensory nerve conduction for CMT1A and CMT2A patients

| | CMT1A | CMT2A | P |
|---|---|---|---|
| Motor NCS | | | |
| Number of patients | 26 | 11 | |
| Median nerves | | | |
| TL^{a} (ms) | 7.8 ± 1.4 | 3.5 ± 0.6^{h} | <0.001 |
| CMAP^{b} (mV) | 8.1 ± 4.6 | 7.4 ± 4.4 | NS^{f} |
| MNCV^{c} (m/s) | 20.4 ± 5.0 | 47.0 ± 6.9^{h} | <0.001 |
| Non-reaction (%) | 3 (12%) | 2 (18%) | NS |
| Ulnar nerves | | | |
| TL^{a} (ms) | 5.6 ± 0.9 | 3.2 ± 0.4^{h} | <0.001 |
| CMAP^{b} (mV) | 9.2 ± 3.0 | 5.1 ± 5.0^{g} | <0.01 |
| MNCV^{c} (m/s) | 19.8 ± 5.6 | 47.9 ± 10.3^{h} | <0.001 |
| Non-reaction (%) | 3 (12%) | 2 (18%) | NS |
| Common peroneal nerves | | | |
| TL^{a} (ms) | 9.0 ± 2.1 | 6.5 | NS |
| CMAP^{b} (mV) | 2.6 ± 2.3 | 0.1 | NS |
| MNCV^{c} (m/s) | 19.5 ± 6.8 | 20.5 | NS |
| Non-reaction (%) | 17 (65%) | 10 (91%) | NS |
| Posterior tibial nerves | | | |
| TL^{a} (ms) | 10.3 ± 4.0 | 13.5 | NS |
| CMAP^{b} (mV) | 4.2 ± 6.1 | 0.1 | NS |
| MNCV^{c} (m/s) | 18.8 ± 6.0 | 26.8 | NS |
| Non-reaction (%) | 12 (46%) | 10 (91%)^{f} | <0.05 |
| Sensory NCS | | | |
| Number of patients | 26 | 11 | |
| Median nerves | | | |
| SNAP^{d} (uV) | 5.5 ± 2.5 | 13.9 ± 12.3 | NS |
| SNAP^{e} (m/s) | 15.8 ± 3.2 | 35.0 ± 3.5^{h} | <0.001 |
| Non-reaction (%) | 19 (73%) | 5 (46%) | NS |
| Ulnar nerves | | | |
| SNAP^{d} (uV) | 4.2 ± 2.6 | 8.4 ± 6.9 | NS |
| SNAP^{e} (m/s) | 16.2 ± 4.3 | 31.4 ± 5.5^{h} | <0.001 |
| Non-reaction (%) | 20 (77%) | 5 (46%) | NS |
| Sural nerves | | | |
| SNAP^{d} (uV) | 8.5 ± 2.1 | 8.4 ± 2.1 | NS |
| SNAP^{e} (m/s) | 18.9 ± 4.1 | 31.9 ± 7.2 | NS |
| Non-reaction (%) | 23 (88%) | 8 (73%) | NS |

^{a}TL: terminal latency;
^{b}CMAP: compound muscle action potential;
^{c}MNCV: motor nerve conduction velocity;
^{d}SNAP: sensory nerve action potential;
^{e}SNCV: sensory nerve conduction velocity; and
NS means that p-value on comparison between CMT1A and CMT2A values is not significant

(4) MRI Findings

1) Measurement and Injury Comparison of Cross-Sectional Areas in Thigh Muscles

The cross-sectional areas of anterior, medial and posterior compartments measured in 26 demyelinating neuropathy patients and 11 axonal neuropathy patients are shown in Table 7. The cross-sectional area was measured on the sectional area which corresponds to the middle of the thigh muscle. The sectional area of the CMT2A patient group was significantly narrower than the CMT1A patient group in all of the anterior, medial and posterior compartments (p<0.05).

TABLE 7
Cross-sectional areas of anterior, medial and posterior compartments
in proximal lower extremities of CMT1A and CMT2A patients

| | CMT1A | CMT2A | P |
|---|---|---|---|
| Number of patients | 26 | 11 | |
| Cross-sectional area | | | |
| Anterior compartments | | | |
| The right | 4837 ± 1126 | 3609 ± 1172 | <0.01 |
| The left | 4795 ± 1211 | 3635 ± 1179 | <0.05 |
| Medial compartments | | | |
| The right | 3656 ± 930 | 2730 ± 1022 | <0.05 |
| The left | 3599 ± 916 | 2634 ± 993 | <0.05 |
| Posterior compartments | | | |
| The right | 2548 ± 613 | 1771 ± 445 | <0.01 |
| The left | 2481 ± 585 | 1792 ± 432 | <0.01 |

^{f}NS means that p-value on comparison between CMT1A and CMT2A values is not significant

Comparing thigh muscle injury between the three compartments, the CMT1A group showed an 85% invasion in posterior compartments which are considerably higher than invasions in anterior (8%) or medial compartment (19%) (Table 8). On the other hand, axonal neuropathy, the CMT2A group, showed 82% and 91% invasions in anterior and posterior compartments, respectively, but showed a slight invasion (73%) in medial compartments. Interestingly, for two clinical symptom-free patients who had been normal in thigh muscle MRC, fatty infiltration was observed on MRI in posterior compartments. Comparing the duration of disease, based on 10 years, i.e., (>10 years) and (<10 years), the case where the disease progresses over 10 years or longer (>10 years), thigh muscle injury level is increased, as compared to the case where the disease progresses below 10 years (<10 years).

TABLE 8
Fatty infiltration frequency of anterior, medial and posterior compartments in proximal lower
extremities for CMT1A and CMT2A patients

| | CMT1A | | | | CMT2A | | | |
|---|---|---|---|---|---|---|---|---|
| | | Duration of disease | | | | Duration of disease | | |
| | Total | ≦10 Years | >10 Years | p | Total | ≦10 Years | >10 Years | p |
| Number of patients | 26 | 9 | 17 | | 11 | 2 | 9 | |
| Compartment | | | | | | | | |
| Anterior | 2 | 0 | 2 | NS | 9 | 1 | 8 | NS |
| | (8%) | (0%) | (12%) | | (82%) | (50%) | (89%) | |
| Medial | 5 | 0 | 5 | NS | 8 | 1 | 7 | NS |
| | (19%) | (0%) | (29%) | | (73%) | (50%) | (78%) | |
| Posterior | 22 | 5 | 17 | NS | 10 | 1 | 9 | NS |
| | (85%) | (56%) | (100%) | | (91%) | (50%) | (100%) | |
| Total | 22 | 5 | 17 | NS | 10 | 1 | 9 | NS |
| | (85%) | (56%) | (100%) | | (91%) | (50%) | (100%) | |

^{f}NS means that p-value on comparison between short disease duration (≦10 years) and long disease duration (>10 years) in CMT groups is not significant

2) Fatty Infiltration Patterns in Different Compartments

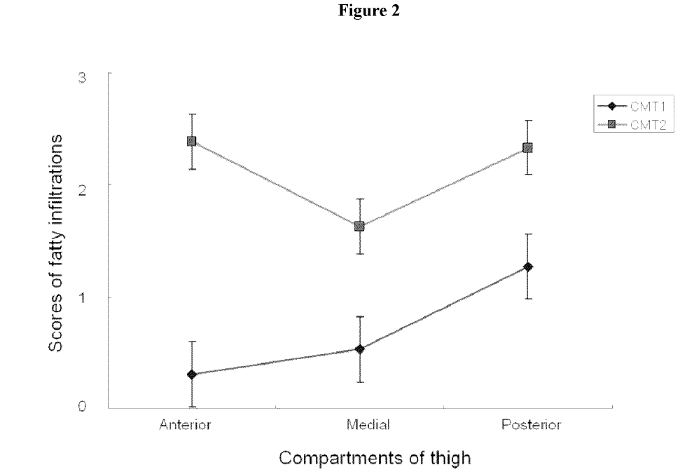

There was a difference between CMT1A and CMT2A patient groups in fatty infiltration behaviors of proximal lower extremity muscles (Table 9). In the CMT1A group, the fatty infiltration level gradually becomes more severe in this order: anterior<medial<posterior compartments. On the other hand, in the CMT2A group, the fatty infiltration level gradually becomes more severe in this order: medial<posterior<anterior compartments (FIG. 2). The CMT2A group showed severe fatty infiltration levels in the all compartments, as compared to the CMT1A group.

The CMT2A group with axonal neuropathy shows statistically significant severe fatty infiltration, as compared to demyelinating neuropathy, the CMT1A group, and in particular, there was marked difference therebetween in anterior compartments. In anterior compartments, the CMT2A group has an average fatty infiltration scale of 2.39, which corresponds to 7-fold or more of the average fatty infiltration scale of the CMT1A group (0.31). In particular, in the vastus lateralis, there was marked difference in average fatty infiltration scale between the CMT2A group (2.82-3.00) and the CMT1A group (0.19-0.31). In medial compartments, the CMT2A group has an average fatty infiltration scale of 1.63, which is about 3-times higher than an average fatty infiltration scale (0.53) of the CMT1A group and in all types of muscles, the average fatty infiltration scale of the CMT2A group is higher than that of the CMT1A group. In posterior compartments, the CMT2A group has an average fatty infiltration of 2.33 which is about 2-fold higher than an average fatty infiltration of the CMT1A group, 1.27, whereas in the biceps femoris, there was no significant difference therebetween.

TABLE 9
Fatty infiltration in three different (i.e., anterior, medial and posterior)
compartments and respective muscles of CMT1A and CMT2A patients

| | CMT1A | CMT2A | p |
|---|---|---|---|
| Number of patients | 26 | 11 | |
| Anterior compartments | 0.31 ± 0.67 | 2.39 ± 1.45 | <0.001 |
| Sartorius | | | |
| The right | 0.88 ± 0.82 | 2.09 ± 1.38 | <0.05 |
| The left | 0.88 ± 0.82 | 2.18 ± 1.33 | <0.01 |
| Rectus femoris | | | |
| The right | 0.15 ± 0.61 | 2.18 ± 1.72 | <0.01 |
| The left | 0.15 ± 0.61 | 2.18 ± 1.72 | <0.01 |
| Vastus medialis | | | |
| The right | 0.15 ± 0.61 | 2.36 ± 1.63 | <0.001 |
| The left | 0.15 ± 0.61 | 2.36 ± 1.63 | <0.001 |
| Vastus intermedius | | | |
| The right | 0.15 ± 0.61 | 2.36 ± 1.63 | <0.001 |
| The left | 0.15 ± 0.61 | 2.36 ± 1.63 | <0.001 |
| Vastus lateralis | | | |
| The right | 0.19 ± 0.80 | 2.82 ± 1.33 | <0.001 |
| The left | 0.31 ± 0.67 | 3.00 ± 1.10 | <0.001 |
| Medial compartments | 0.53 ± 0.48 | 1.63 ± 1.25 | <0.01 |
| Adductor magnus | | | |
| The right | 0.27 ± 0.53 | 1.27 ± 1.01 | <0.01 |
| The left | 0.23 ± 0.51 | 1.36 ± 1.12 | <0.01 |
| Gracilis | | | |
| The right | 0.81 ± 0.57 | 1.19 ± 1.45 | <0.05 |
| The left | 0.81 ± 0.57 | 2.00 ± 1.55 | <0.05 |
| Posterior compartments | 1.27 ± 0.76 | 2.33 ± 1.21 | <0.01 |
| Biceps femoris | | | |
| The right | 1.19 ± 0.75 | 2.09 ± 1.38 | NS |
| The left | 1.23 ± 0.71 | 2.09 ± 1.38 | NS |
| Semitendinosus | | | |
| The right | 1.42 ± 0.86 | 2.63 ± 1.21 | <0.01 |
| The left | 1.35 ± 0.89 | 2.72 ± 1.27 | <0.01 |
| Semimembranosus | | | |
| The right | 1.19 ± 0.90 | 2.18 ± 1.25 | <0.05 |
| The left | 1.27 ± 0.76 | 2.33 ± 1.21 | <0.05 |

NS means that p-value on comparison between CMT1A and CMT2A values is not significant

3) Correlations Between Fatty Infiltration Levels and Lower Extremity Muscle Strength

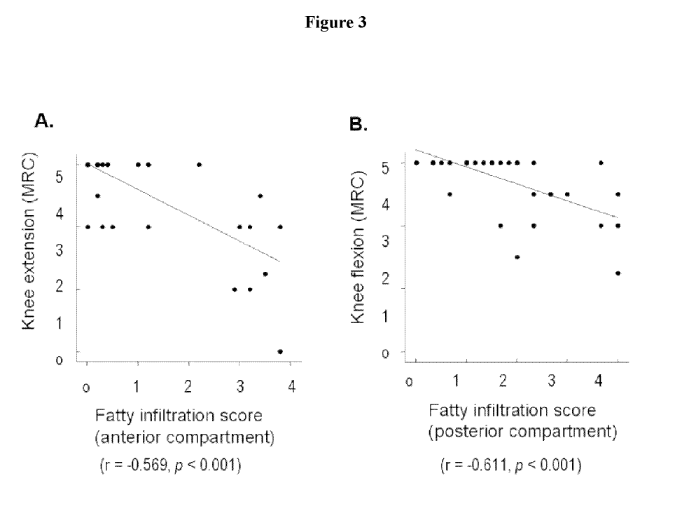

Fatty infiltration level in different compartments of proximal lower extremity muscles and MRC muscle strength grade of the knee joint were compared for CMT patients. The knee joint extension is significantly correlated with anterior compartment fatty infiltration (r=−0.569, p<0.001), and the knee joint flexion is also significantly correlated with posterior compartments fatty infiltration (r=−0.611, p<0.001). Accordingly, as fatty infiltration increases, muscle strength weakness in lower extremities becomes more severe (FIG. 3).

4) Sequential Thigh Muscle Fatty Infiltration Patterns

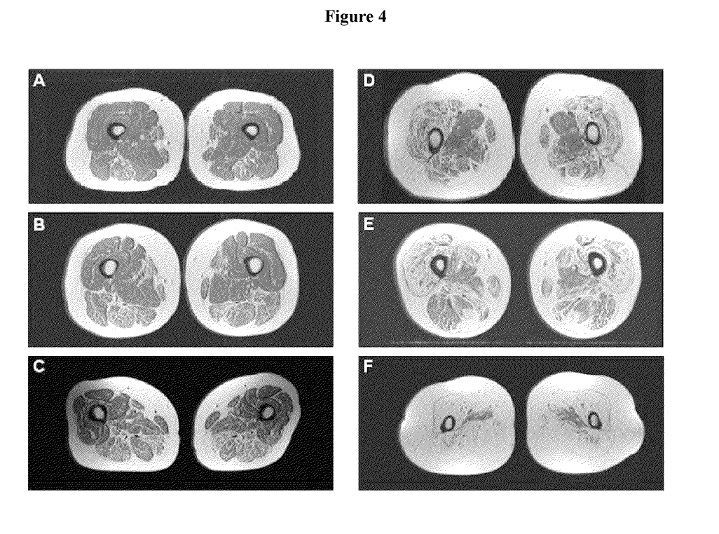

In order to confirm sequential muscle fatty infiltration behaviors depending on the duration of disease, proximal lower extremity MRI results were compared between three CMT1A patients and three CMT2A patients having different disease durations. A 38 year old female patient (FIG. 4A) who suffered from CMT1A for 11 years showed slight fatty infiltration in posterior compartments, and a 63 year old female patient who suffered from CMT1A for 24 years (FIG. 4B) showed more-developed fatty infiltration in posterior compartments and partial fatty infiltration in medial compartments. However, a 86 year old female patient who suffered from CMT1A for 31 years showed marked fatty infiltration in both posterior and medial compartments and relatively preserved anterior compartments (FIG. 4C). Accordingly, fatty infiltration in CMT1A with demyelinating neuropathy increases from posterior compartments to medial compartments in this order. A 14 year old male patient who suffered from the CMT2A disease for 10 years showed medium fatty infiltration in anterior compartments, and slight fatty infiltration in posterior compartments (FIG. 4D). A 36 year old female patient who suffered from the CMT2A disease for 28 years showed severe fatty infiltration in anterior compartments and medium fatty infiltration in posterior compartments (FIG. 4E). A 40 year old female patient who suffer from the CMT2A disease for 31 years showed severe fatty infiltration in all compartments except a part of medial compartments (FIG. 4F). Accordingly, fatty infiltration of CMT2A with axonal neuropathy is progressed in this order: anterior, posterior and medial compartments.

5) Fatty Infiltration of Demyelinating Neuropathy Groups in Respective Compartments

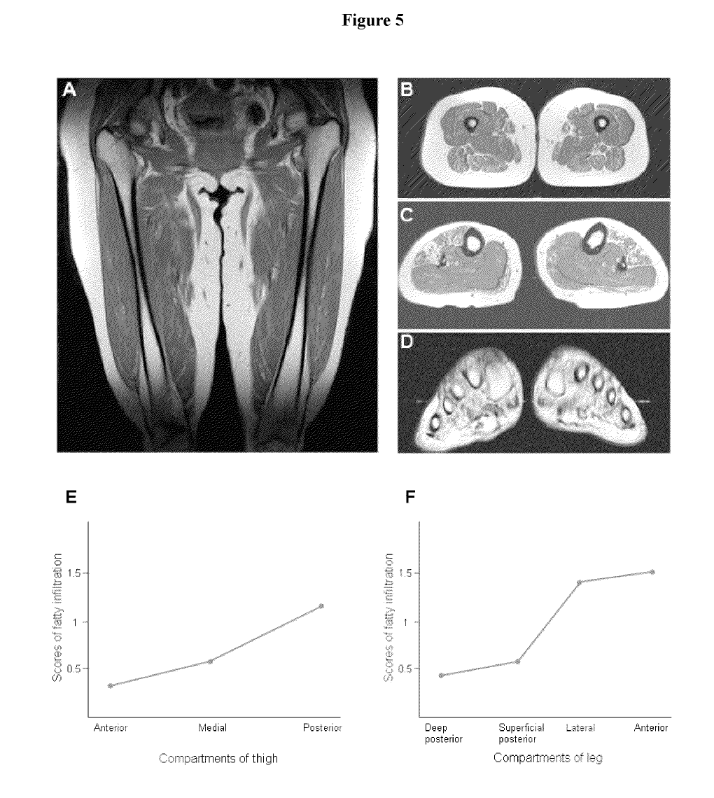

The results of lower extremity MRI performed on CMT1A group with demyelinating neuropathy indicated that muscle injury severity increases in this order: thigh muscles<leg muscles<foot muscles (See FIGS. 5A-5D). This muscle injury severity behavior strongly correlates to the length-dependent injury theory. MRI findings of proximal lower extremity muscles revealed that fatty infiltration severity increases in the order: anterior<medial<posterior compartments and MRI findings of distal lower extremity muscles revealed that fatty infiltration severity increases in the order: deep posterior<superficial posterior<lateral<anterior compartments (FIGS. 5E and 5F). These behaviors mean that muscle injury severity increases, as nerve length increases, which corresponds to nerve length-dependent injury theory.

6) Fatty Infiltration Levels in Respective Compartments of Axonal Neuropathy Groups

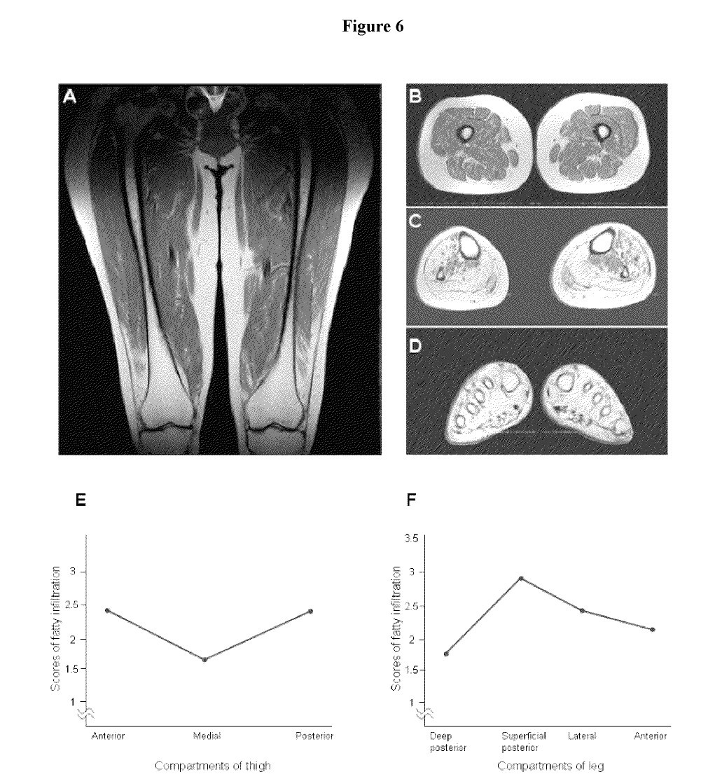

As mentioned above, MRI findings of thigh, leg and foot muscles for the CMT2A group with axonal neuropathy, revealed that fatty infiltration severity increases in the order: thigh muscles<leg muscles<foot muscles, which corresponds to the length-dependent injury theory (FIGS. 6A-6D). However, the results of MRI examination on proximal lower extremities indicated that unlike demyelinating neuropathy, in fatty infiltration severity, anterior compartments are more severe than medial compartments, and anterior compartments are more severe than posterior compartments. In addition, in distal lower extremity muscles, fatty infiltration severity increases in the order: deep posterior<anterior<lateral<superficial posterior, and in particular, is markedly severe in superficial posterior compartments. Muscle injury patterns of axonal neuropathy are different from those of demyelinating neuropathy (FIGS. 6E and 6F).

As apparent from the above description, according to the CMT diagnosis method of the present embodiments, fatty infiltration levels in respective compartments of proximal lower extremity muscles are compared using MRI examination on the proximal lower extremities which has been not conventionally studied, to clearly distinguish CMT1A from CMT2A and thereby realize efficient and useful CMT diagnosis. In addition, some embodiments provide a method for diagnosing CMT1A and CMT2A by evaluation of fatty infiltration levels in respective compartments using MRI examination on distal lower extremities.

Although some of the preferred embodiments have been disclosed for illustrative purposes, those skilled in the art will appreciate that various modifications, additions and substitutions are possible, without departing from the scope and spirit of the technology as disclosed in the accompanying claims.

What is claimed is:

1. A method for diagnosing Charcot-Marie-Tooth disease (CMT), comprising confirming a fatty infiltration increase in one or more compartments of proximal lower extremities by MRI examination on the proximal lower extremities of a patient.

2. A method for diagnosing CMT1A or CMT2A, comprising comparing a fatty infiltration level between anterior compartments, medial compartments and posterior compartments of proximal lower extremity muscles by MRI examination of proximal lower extremities of a patient.

3. The method according to claim 2, wherein the diagnosis of the CMT1A comprises confirming the behavior that fatty infiltration severity in the proximal lower extremity muscle increases in this order: anterior compartments, medial compartments and posterior compartments.

4. The method according to claim 2, wherein the diagnosis of the CMT2A comprises confirming the behavior that the fatty infiltration severity in the proximal lower extremity muscles increases in this order: medial compartments, posterior compartments and anterior compartments.
